(19) United States
(12) Patent Application Publication
(10) Pub. No.: US 2019/0275296 A1
(43) Pub. Date: Sep. 12, 2019

(54) STEERABLE SHEATH FOR INTRAVASCULAR MEDICAL DEVICES

(71) Applicant: Cardiovascular Systems, Inc., New Brighton, MN (US)

(72) Inventors: William G. Besser, Minneapolis, MN (US); Preston L. Grothe, Maple Grove, MN (US); Joseph P. Higgins, Minnetonka, MN (US)

(21) Appl. No.: 16/298,003

(22) Filed: Mar. 11, 2019

Related U.S. Application Data
(60) Provisional application No. 62/641,625, filed on Mar. 12, 2018.

(51) Int. Cl. A61M 25/01

(52) U.S. Cl. CPC A61M 25/0147; A61M 2025/09141; A61M 25/0152; A61M 25/0141

(57) ABSTRACT

Various embodiments of a steerable sheath or catheter are disclosed. The sheath or catheter may comprise a preformed curve or angle proximate the distal end of the sheath or catheter and may comprise either an outer straighter or an inner straightener to deform, or control, the degree of curvature. Other embodiments comprise at least two lumens axially along the sheath or catheter wall that are anchored proximate the distal end of the sheath or catheter and extend proximally to the operator, allowing either pushing or pulling control of the region of the sheath or catheter located distal to the anchoring points of the push or pull wires. The push/pull wires may be used to deform, or at least partially straighten, a precurved section of the sheath or catheter or may be use deform, or at least partially curve a straight section of the sheath.

### CROSS-REFERENCE TO RELATED APPLICATIONS

This application claims the benefit of U.S. Provisional Application No. 62/641,625, filed Mar. 12, 2018 and entitled STEERABLE SHEATH FOR ATHERECTOMY DEVICES AND SYSTEMS, the entirety of which is hereby incorporated by reference.

### STATEMENT REGARDING FEDERALLY SPONSORED RESEARCH OR DEVELOPMENT

Not Applicable

### BACKGROUND OF THE INVENTION

### Field of the Invention

The disclosure relates to devices and methods using sheaths generally with intravascular medical devices and more specifically with atherectomy devices and systems, including but not limited to orbital atherectomy devices and systems.

### Description of the Related Art

It is common to position catheters and/or sheaths to aid in the diagnosis and treatment of various medical disorders such as in the treatment of occlusions, lesions or stenotic material within a blood vessel using, e.g., and without limitation, atherectomy devices and systems including orbital atherectomy devices and systems as well as angioplasty systems. However, there are cases where a strong wire bias exists, such as in regions of vessel curvature, or vessels larger than, e.g., 6 mm. In these cases, the lesion location may not be accessible and, therefore, not treatable using atherectomy and/or angioplasty devices and systems.

Various embodiments of the present invention address these, inter alia, issues.

Moreover, we provide disclosure of the following patents and applications, each of which are assigned to Cardiovascular Systems, Inc., and incorporated herein in their entirety, each of which may comprise systems, methods and/or devices that may be used with various embodiments of the presently disclosed subject matter:

U.S. Pat. No. 6,295,712, “ROTATIONAL ATHERECTOMY DEVICE”;

U.S. Pat. No. 6,494,890, “ECCENTRIC ROTATIONAL ATHERECTOMY DEVICE”;

U.S. Pat. No. 6,132,444, “ECCENTRIC DRIVE SHAFT FOR ATHERECTOMY DEVICE AND METHOD FOR MANUFACTURE”;

U.S. Pat. No. 6,638,288, “ECCENTRIC DRIVE SHAFT FOR ATHERECTOMY DEVICE AND METHOD FOR MANUFACTURE”;

U.S. Pat. No. 5,314,438, “ABRASIVE DRIVE SHAFT DEVICE FOR ROTATIONAL ATHERECTOMY”;

U.S. Pat. No. 6,217,595, “ROTATIONAL ATHERECTOMY DEVICE”;

U.S. Pat. No. 5,554,163, “ATHERECTOMY DEVICE”;

U.S. Pat. No. 7,507,245, “ROTATIONAL ANGIOPLASTY DEVICE WITH ABRASIVE CROWN”;

U.S. Pat. No. 6,129,734, “ROTATIONAL ATHERECTOMY DEVICE WITH RADIALLY EXPANDABLE PRIME MOVER COUPLING”;

U.S. patent application Ser. No. 11/761,128, “ECCENTRIC ABRADING HEAD FOR HIGH-SPEED ROTATIONAL ATHERECTOMY DEVICES”;

U.S. patent application Ser. No. 11/767,725, “SYSTEM, APPARATUS AND METHOD FOR OPENING AN OCCLUDED LESION”;

U.S. patent application Ser. No. 12/130,083, “ECCENTRIC ABRADING ELEMENT FOR HIGH-SPEED ROTATIONAL ATHERECTOMY DEVICES”;

U.S. patent application Ser. No. 12/363,914, “MULTI-MATERIAL ABRADING HEAD FOR ATHERECTOMY DEVICES HAVING LATERALLY DISPLACED CENTER OF MASS”;

U.S. patent application Ser. No. 12/578,222, “ROTATIONAL ATHERECTOMY DEVICE WITH PRE-CURVED DRIVE SHAFT”;

U.S. patent application Ser. No. 12/130,024, “ECCENTRIC ABRADING AND CUTTING HEAD FOR HIGH-SPEED ROTATIONAL ATHERECTOMY DEVICES”;

U.S. patent application Ser. No. 12/580,590, “ECCENTRIC ABRADING AND CUTTING HEAD FOR HIGH-SPEED ROTATIONAL ATHERECTOMY DEVICES”;

U.S. patent application Ser. No. 29/298,320, “ROTATIONAL ATHERECTOMY ABRASIVE CROWN”;

U.S. patent application Ser. No. 29/297,122, “ROTATIONAL ATHERECTOMY ABRASIVE CROWN”;

U.S. patent application Ser. No. 12/466,130, “BIDIRECTIONAL EXPANDABLE HEAD FOR ROTATIONAL ATHERECTOMY DEVICE”;

U.S. patent application Ser. No. 12/388,703, “ROTATIONAL ATHERECTOMY SEGMENTED ABRADING HEAD AND METHOD TO IMPROVE ABRADING EFFICIENCY”;

U.S. Pat. No. 9,468,457, “ATHERECTOMY DEVICE WITH ECCENTRIC CROWN”;

U.S. Pat. No. 9,439,674, “ROTATIONAL ATHERECTOMY DEVICE WITH EXCHANGEABLE DRIVE SHAFT AND MESHING GEARS”;

U.S. Pat. No. 9,220,529, “ROTATIONAL ATHERECTOMY DEVICE WITH ELECTRIC MOTOR”;

U.S. Pat. No. 9,119,661, “ROTATIONAL ATHERECTOMY DEVICE WITH ELECTRIC MOTOR”;

U.S. Pat. No. 9,119,660, “ROTATIONAL ATHERECTOMY DEVICE WITH ELECTRIC MOTOR”;

U.S. Pat. No. 9,078,692, “ROTATIONAL ATHERECTOMY SYSTEM”;

U.S. Pat. No. 6,295,712, “ROTATIONAL ATHERECTOMY DEVICE”;

U.S. Pat. No. 6,494,890, “ECCENTRIC ROTATIONAL ATHERECTOMY DEVICE”;

U.S. Pat. No. 6,132,444, “ECCENTRIC DRIVE SHAFT FOR ATHERECTOMY DEVICE AND METHOD FOR MANUFACTURE”;

U.S. Pat. No. 6,638,288, “ECCENTRIC DRIVE SHAFT FOR ATHERECTOMY DEVICE AND METHOD FOR MANUFACTURE”;

U.S. Pat. No. 5,314,438, “ABRASIVE DRIVE SHAFT DEVICE FOR ROTATIONAL ATHERECTOMY”;

U.S. Pat. No. 6,217,595, “ROTATIONAL ATHERECTOMY DEVICE”;

U.S. Pat. No. 5,554,163, “ATHERECTOMY DEVICE”;

U.S. Pat. No. 7,507,245, “ROTATIONAL ANGIOPLASTY DEVICE WITH ABRASIVE CROWN”;

U.S. Pat. No. 6,129,734, “ROTATIONAL ATHERECTOMY DEVICE WITH RADIALLY EXPANDABLE PRIME MOVER COUPLING”;

U.S. patent application Ser. No. 11/761,128, “ECCENTRIC ABRADING HEAD FOR HIGH-SPEED ROTATIONAL ATHERECTOMY DEVICES”;

U.S. patent application Ser. No. 11/767,725, “SYSTEM, APPARATUS AND METHOD FOR OPENING AN OCCLUDED LESION”;

U.S. patent application Ser. No. 12/130,083, “ECCENTRIC ABRADING ELEMENT FOR HIGH-SPEED ROTATIONAL ATHERECTOMY DEVICES”;

U.S. patent application Ser. No. 12/363,914, “MULTI-MATERIAL ABRADING HEAD FOR ATHERECTOMY DEVICES HAVING LATERALLY DISPLACED CENTER OF MASS”;

U.S. patent application Ser. No. 12/578,222, “ROTATIONAL ATHERECTOMY DEVICE WITH PRE-CURVED DRIVE SHAFT”;

U.S. patent application Ser. No. 12/130,024, “ECCENTRIC ABRADING AND CUTTING HEAD FOR HIGH-SPEED ROTATIONAL ATHERECTOMY DEVICES”;

U.S. patent application Ser. No. 12/580,590, “ECCENTRIC ABRADING AND CUTTING HEAD FOR HIGH-SPEED ROTATIONAL ATHERECTOMY DEVICES”;

U.S. patent application Ser. No. 29/298,320, “ROTATIONAL ATHERECTOMY ABRASIVE CROWN”;

U.S. patent application Ser. No. 29/297,122, “ROTATIONAL ATHERECTOMY ABRASIVE CROWN”;

U.S. patent application Ser. No. 12/466,130, “BIDIRECTIONAL EXPANDABLE HEAD FOR ROTATIONAL ATHERECTOMY DEVICE”; and

U.S. patent application Ser. No. 12/388,703, “ROTATIONAL ATHERECTOMY SEGMENTED ABRADING HEAD AND METHOD TO IMPROVE ABRADING EFFICIENCY”

U.S. patent application Ser. No. 13/681,398, “ROTATIONAL ATHERECTOMY DEVICE WITH A SYSTEM OF ECCENTRIC ABRADING HEADS”; and

U.S. patent application Ser. No. 15/906,067, “ECCENTRIC SYSTEM OF ABRASIVE ELEMENTS WITH EQUAL MASS FOR ROTATIONAL ATHERECTOMY

### BRIEF SUMMARY OF THE INVENTION

Various embodiments of a steerable sheath or catheter are disclosed. The sheath or catheter may comprise a preformed curve or angle proximate the distal end of the sheath or catheter and may comprise either an outer straighter or an inner straightener to deform, or control, the degree of curvature. Other embodiments comprise at least two lumens axially along the sheath or catheter wall that are anchored proximate the distal end of the sheath or catheter and extend proximally to the operator, allowing either pushing or pulling control of the region of the sheath or catheter located distal to the anchoring points of the push or pull wires. The push/pull wires may be used to deform, or at least partially straighten, a precurved section of the sheath or catheter or may be use deform, or at least partially curve a straight section of the sheath. In some cases, the curving point may be a weakened portion of the sheath or catheter wall, or may comprise a shape memory material with at least one preset position. Further, the push/pull wires may be configured to transmit heat to a curveable section that is responsive to changes in temperature, thereby enabling curving, or straightening, when activated by heat.

The figures and the detailed description which follow more particularly exemplify these and other embodiments of the invention.

### BRIEF DESCRIPTION OF THE SEVERAL VIEWS OF THE DRAWINGS

FIG. 1A is a cutaway view of a prior art device.

FIG. 1B is a cutaway view of one embodiment of the present invention illustrated as an angled or J-shape activated steerable sheath within a blood vessel and proximate a lesion.

FIG. 2 is a side view of one embodiment of the present invention.

FIG. 3A is a side view of one embodiment of the present invention prior to deployment.

FIG. 3B is a side view of the embodiment of FIG. 3A in a deployed configuration.

FIG. 4A is a side view of one embodiment of the present invention prior to deployment.

FIG. 4B is a side view of the embodiment of FIG. 4A in a deployed configuration.

FIG. 5 is a front view of first, second and inner lumens comprising one embodiment of the steerable sheath of the present invention.

FIG. 6A is a side cutaway view of one embodiment of the present invention.

FIG. 6B is a side cutaway view of one embodiment of the present invention.

FIG. 6C is a side cutaway view of one embodiment of the present invention.

FIG. 6D is an end view of one embodiment of the present invention.

FIG. 7 is a side cutaway view of one embodiment of the present invention.

FIG. 8 is a side cutaway view of one embodiment of the present invention.

FIG. 9 is a side cutaway view of one embodiment of the present invention.

FIG. 10A is a side cutaway view of one embodiment of the present invention.

FIG. 10B is a side cutaway view of one embodiment of the present invention.

FIG. 10C is a side cutaway view of one embodiment of the present invention.

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

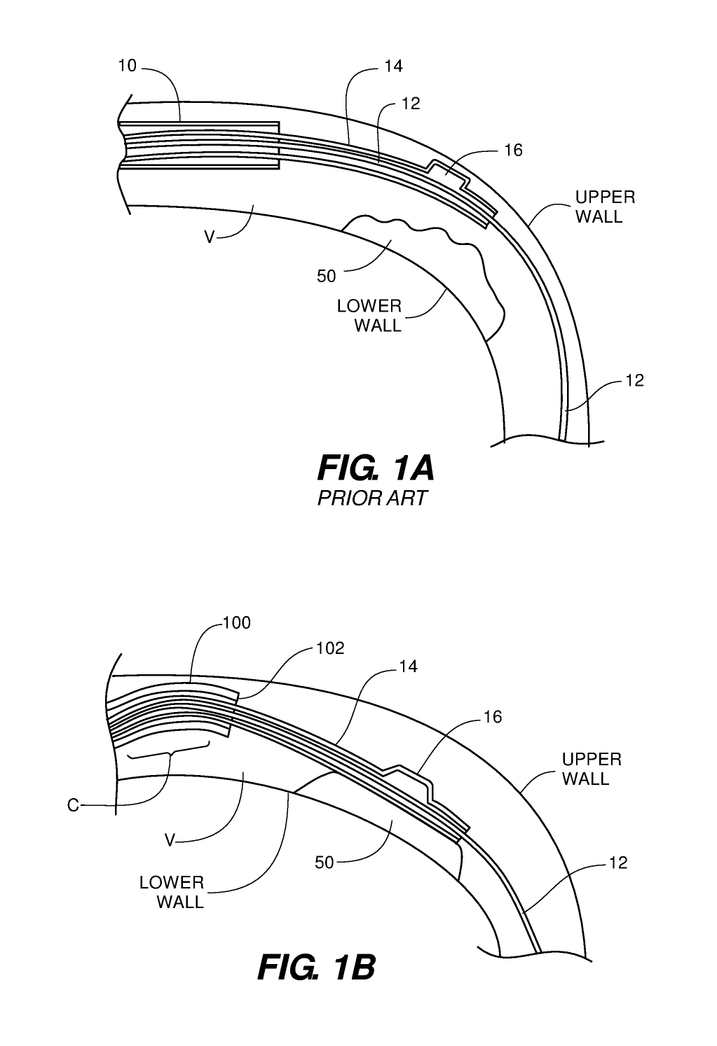

FIG. 1A illustrates a known catheter or sheath 10 comprising a lumen extended partially within a blood vessel V. Guide wire 12 and drive shaft 14 comprising a lumen along which drive shaft is translated and/or rotated, an abrasive element 16, extend distally out of the distal end of catheter 10. As shown, the blood vessel V comprises a curve, with an occlusion or lesion 50 at the bottom wall of the curve within the vessel V. Because the catheter 10 is unable to bend to accommodate the curvature of the vessel V, the guide wire 12 and drive shaft 14 curve along the upper wall of the vessel V, effectively missing the lesion 50.

FIG. 1B shows the blood vessel V and lesion 50 of FIG. 1A. However, catheter 100 comprises a curvature C proximate the distal end 102. Curvature C enables, as illustrated, the guide wire 12 and drive shaft 14 with associated abrasive element 16 to reach further down within the vessel V, i.e., closer to the lower wall and away from the upper wall and lesion 50 than the prior art device of FIG. 1A. At this point, the abrasive element 16 may contact lesion 50 and drive shaft 14 may be rotated to enable abrasive element 16 to abrade the lesion 50.

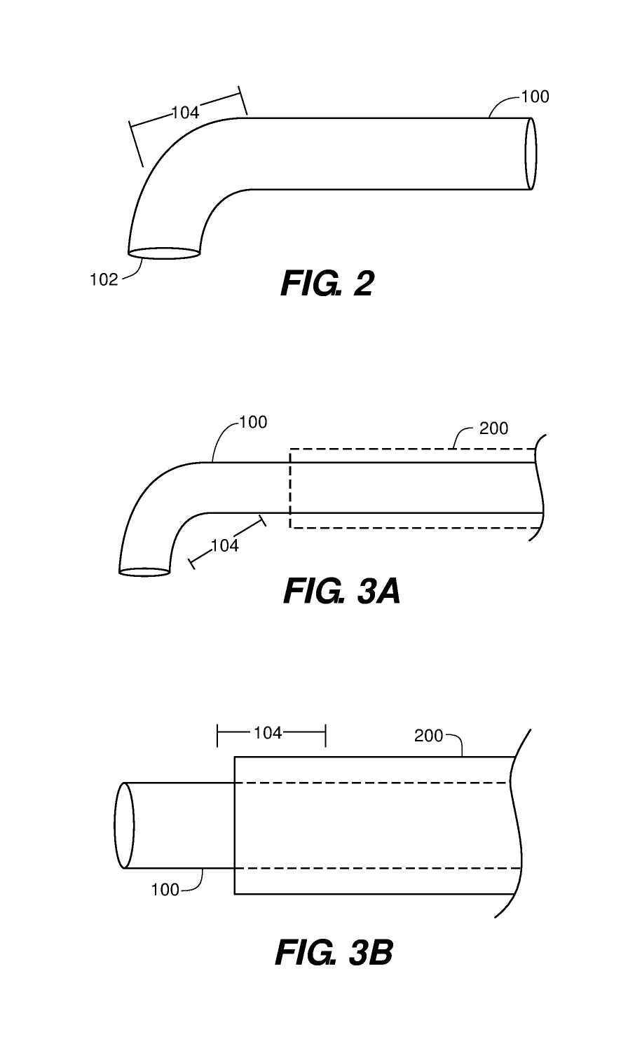

FIG. 2 illustrates one embodiment of a steerable catheter 100, comprising a precurved section 104 resulting in a curvature of the catheter 100 in the precurved section 104. Precurved section 104 is preferably proximate the distal end 102 of catheter 100.

FIGS. 3A and 3B illustrate the steerable catheter 100 of FIG. 2, with an outer straightener 200. Outer straightener 200 comprises a lumen L within which the steerable catheter 100 is at least partially disposed and wherein the steerable catheter 100 may be translated and/or rotated relative to the steerable catheter 100. As illustrated, advancement of the outer straightener 200 over the precurved section 104 of steerable catheter effectively straightens, or deforms, the precurved section 104. The deformation or straightening of preformed section 104 of steerable catheter 100 may also be achieved by retraction of the steerable catheter 100 relative to the outer straightener 104. As may be appreciated, the undeformed or precurved curvature of precurved section 104 may be controlled by an incremental coverage of the precurved section 104 by the outer straightener 200 by the methods described above. As a result, the precurved section 104 may comprise a curvature that ranges from a maximum curvature at its undeformed precurved configuration, as in FIG. 2, to a fully straightened deformed configuration as in FIG. 3B. FIG. 3A illustrates an incremental deformation of precurved section 104 by outer straightener 200, wherein the curvature of precurved section 104 is not at its maximum nor is it fully straightened.

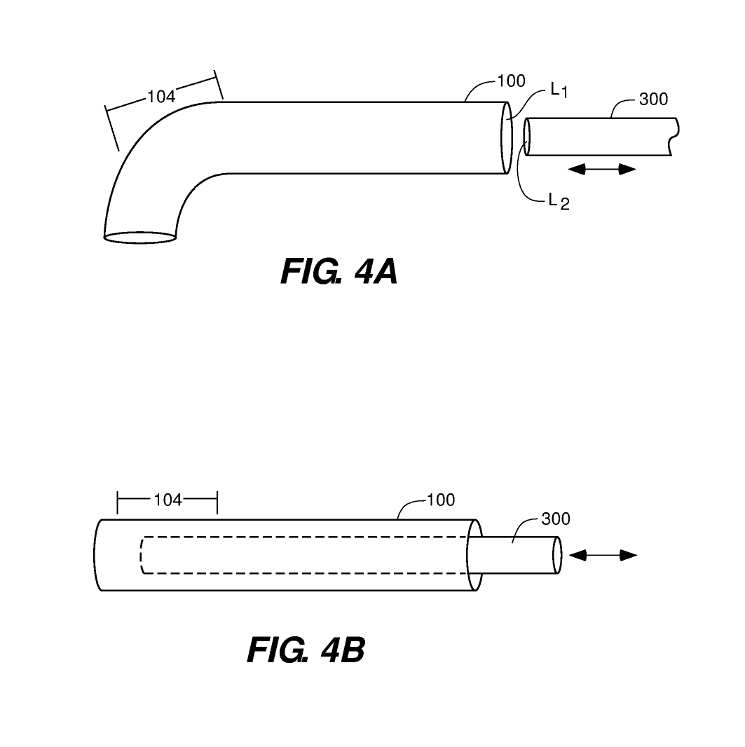

The elements of FIGS. 3A and 3B may be reversed as shown in FIGS. 4A and 4B. Thus, steerable catheter 100 comprises a precurved section 104 as described above and a lumen L₁ therethrough. Inner straightener 300, comprising lumen L₂ therethrough, is configured to be advanceable and/or rotateable within lumen L₁. Lumen L₂ enables the guide wire 12 and/or the drive shaft 14 of FIG. 1B to translate and/or rotate therein. As the inner straightener 300 is advanced to and in some cases incrementally through the precurved section 104 of steerable catheter 100, the curvature of precurved section 104 may be deformed from a maximum undeformed curvature to a minimum curvature, i.e., a straightened deformed shape as in FIG. 4B, and any incremental curvature therebetween. The degree or increment of advancement of the inner straightener 300 relative to the precurved section 104 determines the deformation of precurved section 104 and, therefore, the curvature thereof. The skilled artisan will now appreciate the incremental steering capabilities of the embodiments shown in FIGS. 3A/3B and 4A/4B.

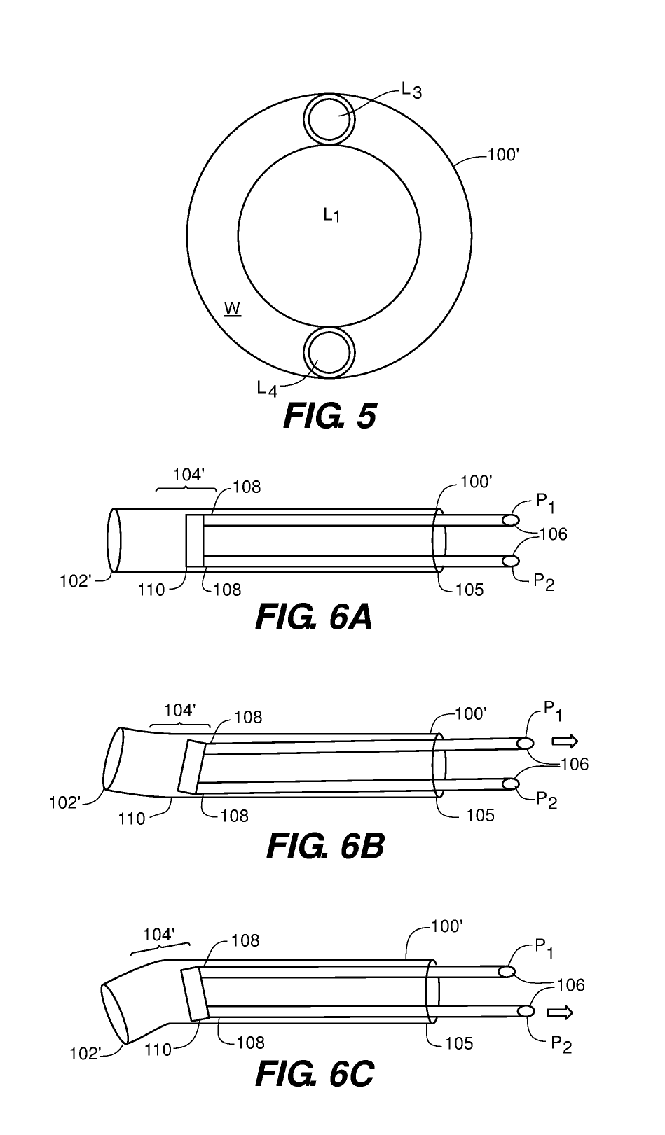

Turning now to FIGS. 5-7C, an alternate steerable catheter 100′ is illustrated. FIG. 5 is an and view showing central lumen L₁, catheter wall W with a first wall lumen L₃ and a second wall lumen L₄ spaced apart from each other, the lumens L₃ and L₄ are formed within catheter wall W and extend at least partially along the length of catheter 100′. Preferably, lumens L₃ and L₄ are spaced at a rotational angle of 180 degrees from each other as shown in FIG. 5. However, alternate rotational angle spacings between L₃ and L₄, e.g., greater or less than 180 degrees, may also be used and are within the scope of the present invention. Lumen L₁ extends longitudinally through the central portion of catheter 100′ and is defined by wall W. Lumens L₃ and L₄ formed within wall S and extend from the proximal end 105 of steerable catheter 100′ to a location or terminus that is located proximal to the distal end 102′ of the catheter 100′. Stated differently, L₃ and L₄ do not extend all the way through the catheter 100′.

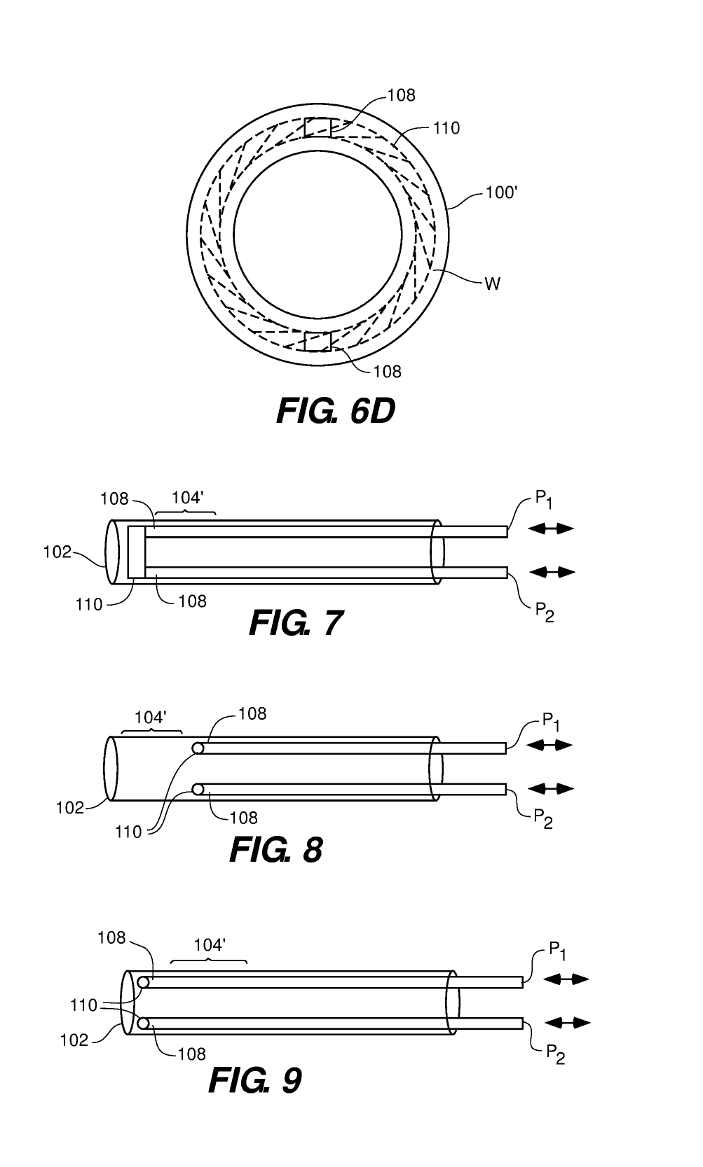

FIGS. 6A-6B show steerable catheter 100′ with curvable section 104′ disposed proximate the distal end 102′ of catheter 100′. A first pull wire P₁ is disposed in lumen L₃ and a second pull wire P₂ is disposed in lumen L₄. Accordingly, pull wires P₁ and P₂ are rotationally spaced from each other in accordance with the rotational angle separating lumens L₃ and L₄. Pull wires P₁ and P₂ each comprise a proximal end 106 and a distal end 108 and are shown extending proximally away from the proximal end 105 of steerable catheter 100′.

As shown, the distal ends 108 of pull wires P₁ and P₂ are attached to a pull ring 110 that may also be disposed within wall W of steerable catheter 100′. In the illustrated embodiment, pull ring 110 and distal ends 108 of pull wires P₁, P₂ are positioned proximal to the curvable section 104′. As shown in FIG. 6B, pulling pull wire P₁ in the proximal direction may cause the curvable section 104′ to curve in an upward direction while pulling pull wire P₂ in the proximal direction may cause curvable section 104′ to curve in a downward direction as in FIG. 6C.

Pull ring 110 of FIGS. 6A-6C is shown as viewed from the distal end 102′ of catheter 100′ in FIG. 6D as embedded in wall W and comprising a circular shape, with attachment to distal ends 108 of pull wires.

Alternatively, as in FIG. 7, pull ring 110 may be disposed distal to the curveable section 104′, so that pulling pull wires P₁ or P₂ will have similar curving effects as shown in FIGS. 6B and 6C.

Still more alternatively, as shown in FIGS. 8 and 9, the attachment of the distal ends 108 to the catheter 100′ proximate the distal end 102 of catheter 100′ need not comprise a fully circular pull ring 110 as in FIGS. 6A-6D. Instead, as shown a partial, e.g., semicircular or other shape pull ring 110′ may be provided within wall W to attach to the distal ends 108 of pull wires P₁ and P₂. Still more alternatively, distal ends 108 of pull wires P₁ and P₂ may simply be embedded within the material of wall W distal to lumens L₃ and L₄. FIG. 8 shows the distal ends 108 of pull wires P₁ and P₂ terminating on the proximal side of curvable section 104′.

Still further, partial pull rings 110′ may be disposed within wall W of catheter 100′ on the distal side of curvable section 104′. As described above, an alternative embodiment may comprise the distal ends of pull wires P₁ and P₂ to simply be embedded in the wall W material distal to lumens L₃ and L₄.

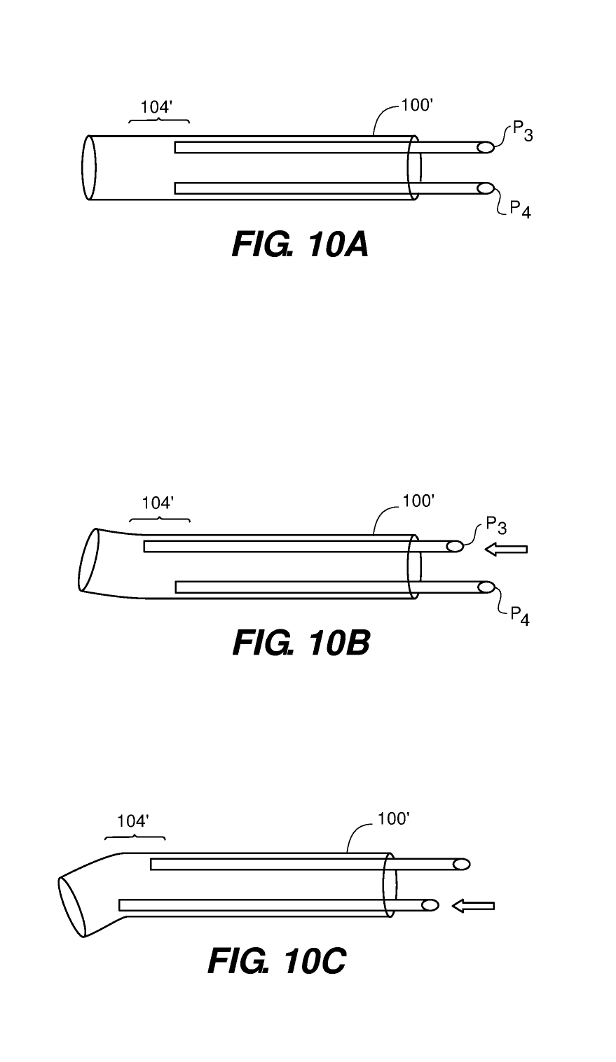

FIGS. 10A-10B illustrate an embodiment of steerable catheter 100′ comprising push wires P₃ and P₄ rather than pull wires P₁ and P₂ of the previously described embodiments. However, the skilled artisan will readily recognize that a wire of sufficient stiffness may be used to both pull and to push, as shown by the double-sided arrows of FIGS. 7-9.

Thus, as in FIG. 10B, pushing top push wire P₃ results in curvable section 104′ curving away from the straightened undeformed configuration of FIG. 10A while pushing bottom wire P₄ results in curvable section 104′ curving in a different direction away from the straightened configuration of FIG. 10A.

As described previously, whether pull wires P₁, P₂, push wires P₃, P₄ or a combination of push/pull wires are employed, the distal ends of the wires may be attached either on the proximal side of the curvable region 104′ or on the distal side of curvable region 104′.

We note further that pull P₁, P₂ and/or push wires P₃, P₄ may be of same or similar length, or may be unequal in length. In certain embodiments, therefore, the terminus or location of the distal end 108 of one or more push and/or pull wires comprise variable distance(s) from the distal end 102′ of catheter 100′.

Moreover, the skilled artisan will recognize that more than two lumens in catheter wall W may be used, wherein each lumen may either be equally rotationally spaced from adjacent lumens or unequally rotationally spaced from adjacent lumens.

The precurving of precurved section 104 may be achieved by heat treatment to provide an undeformed curvature that may be straightened by degrees or increments as discussed herein. This straightening may be achieved using the straighteners 200, 300 or the pull P₁, P₂ and/or push P₃, P₄ wires discussed herein.

Moreover, a shape memory material such as Nitinol may be used to develop a preset curved configuration for precurved section 104.

Curvable section 104′ may be formed by creating a weakened region along the catheter wall W using, e.g., striations or slits or thinner or more flexible material to enable and encourage curving at the curvable section 104.

Further, the precurved section 104 may be moved to a preset curvature, or a range of curvatures, by application of heat transmitted from an external heat generator through one or more than one of the push and/or pull wires. This embodiment may comprise a shape memory alloy to facilitate achieving preset curvatures activated by the application of varying degrees of heat.

According to the above description, the following embodiments, among others, are possible for a steerable sheath or sheath:

1. A steerable catheter or sheath comprising a precurved section formed by heat treatment and/or shape memory alloy such as Nitinol.

2. The precurved sheath of (1) wherein the precurved section may be straightened incrementally away from the curvature of the precurved section.

3. The precurved sheath of (1)-(2), wherein the precurved section may be straightened by translation of an outer straightener or an inner straightener relative to the catheter and precurved section.

4. The precurved sheath of (1)-(3) wherein the precurved section may be straightened by pull and/or push wires.

5. The precurved sheath or catheter of (4) wherein the pull and/or push wires may be used to move the steerable catheter or sheath from one undeformed shape to a deformed shape.

6. A steerable sheath comprising a curvable section, wherein the curvable section may be deformed to a curved configuration.

7. The steerable sheath of (6), wherein the deformation to a curved configuration is achieved by a weakened or stressed or striated section on the steerable catheter or sheath.

8. The steerable sheath of (6)-(7), wherein the deformation to a curved configuration is achieved by manipulation of pull and/or push wires.

9. The steerable sheath of (1)-(8), wherein a deformation from an undeformed configuration to a deformed configuration is achieved using push and/or pull wires disposed in lumens defined in the wall of the steerable catheter.

10. The steerable sheath of (1)-(9), wherein the push and/or pull wires comprise distal ends that are located on the proximal side of the precurved section or curvable section.

11. The steerable sheath of (1)-(10), wherein the push and/or pull wires comprise distal ends that are located on the distal side of the precurved section or curvable section.

12. The steerable sheath of (1)-(11) wherein the push and/or pull wires are anchored at their distal ends at a point distal to the terminus of the first and second wall lumens.

The descriptions of the embodiments and their applications as set forth herein should be construed as illustrative, and are not intended to limit the scope of the disclosure. Features of various embodiments may be combined with other embodiments and/or features thereof within the metes and bounds of the disclosure. Upon study of this disclosure, variations and modifications of the embodiments disclosed herein are possible, and practical alternatives to and equivalents of the various elements of the embodiments will be understood by and become apparent to those of ordinary skill in the art. Such variations and modifications of the embodiments disclosed herein may be made without departing from the scope and spirit of the invention. Therefore, all alternatives, variations, modifications, etc., as may become to one of ordinary skill in the art are considered as being within the metes and bounds of the instant disclosure.

What is claimed is:

1. A steerable catheter having a proximal end, a distal end and a wall defining a central lumen extending from a proximal end to a distal end of the steerable catheter, comprising:
a first wall lumen defined in the wall, extending from the proximal end of the steerable to a terminus located proximal of the distal end of the steerable catheter;
a second wall lumen defined in the wall, extending from the proximal end of the steerable to a terminus located proximal of the distal end of the steerable catheter, wherein the first wall lumen and the second wall lumen are rotationally separated from each other;
a first push and/or pull wire disposed in the first wall lumen and extending out of the proximal end of the catheter and comprising a distal end;
a second push and/or pull wire disposed in the second wall lumen and extending out of the proximal end of the catheter and comprising a distal end;
wherein the distal ends of the first and second push and/or pull wires are anchored at a location distal to the terminus of the first and second wall lumens.

2. The steerable catheter of claim 1, wherein the first wall lumen and the second wall lumen are rotationally separated by a rotational angle of 180 degrees.

3. The steerable catheter of claim 1, wherein the first wall lumen and the second wall lumen are rotationally separated by a rotational angle of less than 180 degrees.

4. The steerable catheter of claim 1, wherein the first wall lumen and the second wall lumen are rotationally separated by a rotational angle of greater than 180 degrees.

5. The steerable catheter of claim 1, wherein the distal ends of the first and second push and/or pull wires are anchored by embedding the distal ends within the wall of the catheter.

6. The steerable catheter of claim 1, further comprising a ring embedded in the wall of the catheter, wherein the distal ends of the first and second push and/or pull wires are anchored by attachment to the ring.

7. The steerable catheter of claim 1, further comprising a curvable section located proximal of the distal end of the steerable catheter.

8. The steerable catheter of claim 7, wherein the curvable section is formed in part by slits or striations along the wall of the steerable catheter.

9. The steerable catheter of claim 1, further comprising a precurved section located proximal of the distal end of the steerable catheter.

10. The steerable catheter of claim 9, wherein the precurved section is formed by one of the group consisting of: heat treatment; a memory shape alloy; and activated by heat.

11. The steerable catheter of claim 9, wherein the first and second push and/or pull wires are configured to transmit heat from a heat generation source attached to the proximal ends of the first and second push and/or pull wires.

12. The steerable catheter of claim 7, wherein the distal ends of the first and second push and/or pull wires are anchored at a location on the proximal side of the curvable section.

13. The steerable catheter of claim 7, wherein the distal ends of the first and second push and/or pull wires are anchored at a location on the distal side of the curvable section.

14. The steerable catheter of claim 9, wherein the distal ends of the first and second push and/or pull wires are anchored at a location on the proximal side of the precurved section.

15. The steerable catheter of claim 9, wherein the distal ends of the first and second push and/or pull wires are anchored at a location on the distal side of the precurved section.

16. A precurved steerable catheter, comprising:
a precurved section located proximal to the distal end of the steerable catheter;
an outer straightener comprising a lumen adapted to receive the precurved steerable catheter and translate independently over the steerable catheter, wherein advancement of the outer straightener over at least a portion of the precurved section deforms the precurved section to a deformed configuration.

17. The precurved steerable catheter of claim 16, wherein the deformed configuration comprises a straight configuration.

18. The precurved steerable catheter of claim 16, wherein the deformed configuration comprises a curved configuration.

19. A precurved steerable catheter, comprising:
a precurved section located proximal to the distal end of the steerable catheter;
an inner straightener adapted to translate within a central lumen of the precurved steerable catheter;
wherein advancement of the inner straightener within at least a portion of the precurved section deforms the precurved section to a deformed configuration.

20. The precurved steerable catheter of claim 19, wherein the deformed configuration comprises a straight configuration.

21. The precurved steerable catheter of claim 19, wherein the deformed configuration comprises a curved configuration.
